(19) United States
(12) Patent Application Publication
(10) Pub. No.: US 2025/0152642 A1
(43) Pub. Date: May 15, 2025

(54) NASAL ANTI-CANCER THERAPIES

(71) Applicant: ORGENESIS INC., GERMANTOWN, MD (US)

(72) Inventors: Oren Moskovich, Rishon LeZion (IL); Nitzan LEVY, Ness Ziona (IL); Ariel ANGEL, Rishon LeZion (IL)

(73) Assignee: ORGENESIS INC., GERMANTOWN, MD (US)

(21) Appl. No.: 18/727,113

(22) PCT Filed: Jan. 5, 2023
(86) PCT No.: PCT/US2023/060160

§ 371 Date: Jul. 8, 2024

Related U.S. Application Data
(60) Provisional application No. 63/385,222, filed on Nov. 29, 2022.
(60) Provisional application No. 63/370,189, filed on Aug. 2, 2022.
(60) Provisional application No. 63/296,953, filed on Jan. 6, 2022.

(51) Int. Cl. A61K 35/761; A61K 9/00; A61K 9/50; A61P 35/00; C12N 5/0775

(52) U.S. Cl. CPC A61K 35/761; A61K 9/0043; A61K 9/5068; A61P 35/00; C12N 5/0663; C12N 2510/00; C12N 2710/10332

(57) ABSTRACT

Provided herein are methods for treating cancer comprising intranasal administration of an oncolytic virus or an oncolytic virus-based vector, comprised in a mesenchymal stem cell (MSC), an MSC-derived EV, or in an MSC-derived bioxome. Further, the MSCs, MSC-derived EVs, or MSC-derived bioxomes disclosed herein may be used for treating a central nervous system (CNS) related disorder.

### FIELD OF THE INVENTION

Provided herein are methods for treating cancer comprising intranasal administration of an oncolytic virus or an oncolytic virus-based vector, comprised in a mesenchymal stem cell (MSC), an MSC-derived extracellular vesicle (EV), or in an MSC-derived bioxome. Further, the MSCs, MSC-derived EVs, or MSC-derived bioxomes disclosed herein may be used for treating a central nervous system (CNS) related disorder.

### BACKGROUND

An oncolytic virus is a virus that preferentially infects and/or kills cancer cells. As the infected cancer cells are destroyed by oncolysis, they often release new infectious virus particles or virions to help destroy the remaining tumor. Oncolytic viruses are thought not only to cause direct destruction of the tumor cells, but also to stimulate host anti-tumor immune system responses.

International PCT Application Publication WO 2019/043282 describes a combination product for the treatment of cancer, which comprises a modified mesenchymal stem cell, and an antigenic substance.

International PCT Application Publication WO 2019/198068, to the present applicant, describes an artificial “bioxome” particle comprising a cell membrane component derived from a selected cellular or extracellular source, engineered to carry a cargo and designed to undergo fusion with a target cell.

A First-in-Human, First-in-Child trial of autologous MSCs carrying the oncolytic virus ICOVIR-5 in patients with advanced tumors was recently reported (Mol Ther. 2020, 28 (4): 1033-1042).

Despite much progress in these technologies, cancer remains a major prophylactic and therapeutic challenge. Potent combinatorial anti-cancer therapies, employing a multitude of different technologies, are required, to defeat this disease.

### SUMMARY

The present invention provides, in one aspect, a method for treating cancer in a patient, the method comprising the step of administering to the patient a first active agent, the first active agent selected from the group consisting of an oncolytic virus and an oncolytic virus-based vector, wherein the first active agent: (i) is comprised in a mesenchymal stem cell (MSC), in an MSC-derived EV, or in an MSC-derived bioxome, and (ii) is administered intranasally to the patient.

In certain embodiments, the cancer is CNS cancer. In certain embodiments, the cancer is brain cancer. In certain embodiments, the brain cancer is glioblastoma. In certain embodiments, the brain cancer is glioblastoma multiforme (GBM). In certain embodiments, the brain cancer is glioma.

In certain embodiments, the cancer is a solid tumor. In certain embodiments, the cancer is a primary tumor. In certain embodiments, the cancer is a metastatic tumor. In certain embodiments, the cancer is a solid metastatic tumor.

In certain embodiments, the first active agent is an oncolytic virus-based vector. In certain embodiments, the oncolytic virus-based vector is selected from the group consisting of an Adenovirus-based vector, an HSV-based vector, a VACV-based vector, a VSV-based vector, a Poliovirus-based vector, a Reovirus-based vector, a Senecavirus-based vector, an Echovirus-based vector, a SFV-based vector, a Maraba virus-based vector, and an Enterovirus-based vector. In certain embodiments, the oncolytic virus-based vector is selected from the group consisting of ICOVIR-5, a Herpes simplex virus type 1 mutant 1716 (HSV-1716), Oncorine (H101), Onyx-15 (dl1520), ColoAd1, Talimogene laherparepvec (T-VEC), GL-ONC1, CV706, and GLV-1h68. In certain embodiments, the oncolytic virus-based vector is ICOVIR-5.

In certain embodiments, the first active agent is an oncolytic virus. In certain embodiments, the oncolytic virus is selected from the group consisting of a Herpes simplex virus (HSV), an Adenovirus, a Vaccinia virus (VACV), a Vesicular stomatitis virus (VSV), a Poliovirus, a Reovirus, a Senecavirus, an Echovirus, a Semliki Forest virus (SFV), a Maraba virus, and an Enterovirus.

In certain embodiments, the first active agent is comprised in an MSC. In certain embodiments, the first active agent is comprised in an MSC-derived EV. In certain embodiments, the first active agent is comprised in an MSC-derived bioxome.

In certain embodiments, in the MSC: (i) Toll-like receptors selected from the group consisting of TLR1, TLR2, TLR4, TLR5, TLR6, TLR7, TLR8, TLR9, and TLR10, gene or protein, are not inhibited, (ii) the MyD88 gene or protein, are not inhibited, and/or (iii) the MAVS gene or protein, are not inhibited.

In certain embodiments, in the MSC: (i) Toll-like receptors selected from the group consisting of TLR1, TLR2, TLR4, TLR5, TLR6, TLR7, TLR8, TLR9, and TLR10, gene or protein, are inhibited, (ii) the MyD88 gene or protein, are inhibited, and/or (iii) the MAVS gene or protein, are inhibited.

In certain embodiments, in the MSC, TLR4, TLR9, and/or the MyD88 gene or protein, are inhibited.

In certain embodiments, in the MSC, the MAVS gene or protein, are inhibited

In certain embodiments, the first active agent is administered intranasally to the brain of the patient. In certain embodiments, the first active agent is applied to the olfactory nerve cells of the patient. In certain embodiments, the first active agent is administered intranasally by a device selected from the group consisting of an instillation catheter, a dropper, a spray, a squeeze bottle, a pump spray, a compressed air nebulizer, a metered-dose inhaler, and an insufflator. In certain embodiments, the first active agent is administered by a controlled particle dispersion device.

The present invention further provides, in another aspect, a mesenchymal stem cell (MSC), wherein in the MSC the MAVS gene and/or protein is inhibited, an EV derived from the MAVS-inhibited MSC, or a bioxome derived from the MAVS-inhibited MSC.

The present invention further provides, in another aspect, a method for the production of a substantially pure population of modified mesenchymal stem cells, comprising the steps of: (i) obtaining a sample of mesenchymal stem cells, (ii) culturing the mesenchymal stem cells obtained in step (i), and (iii) inhibiting the MAVS gene or protein, or of a gene or protein regulated by the MAVS gene or protein, in the cells cultured in step (ii).

The present invention further provides, in another aspect, a method for treating a central nervous system (CNS) related disorder, the method comprising administering to the patient a mesenchymal stem cell (MSC), an MSC-derived EV, or an MSC-derived bioxome, wherein said administration is intranasal administration to the patient. In certain embodiments, the MSC, MSC-derived EV, or MSC-derived bioxome comprises an oncolytic virus or an oncolytic virus-based vector.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a demonstration of Oncolytic viral infection induced cytopathic effect in mouse MSC (WT) 12 days post infection.

FIG. 2 is a graphic demonstration of the effect of conditioned media (CM) containing Oncolytic viral infection (collected from WT infected cells) in the reduction of the proliferation of GL261 cells, 48 and 96 h post treatment; cell number is presented as relative to UT cells. UT=untreated

FIG. 3 shows IVIS scan of GL261 Syngeneic Murine Glioblastoma model mice showing glioblastoma GL261 luciferase labeled cells on day 15, administered with different cell doses. Low dose: 0.1×10⁶ cells; Mid dose: 0.2×10⁶ cells; High dose: 0.5×10⁶ cells.

FIGS. 4A-4B are H&E staining images demonstrating tumor model establishment (brain cranial cross section) on day 29 in mock (FIG. 4A) or 0.5×10⁶ GL261 (FIG. 4B) treated animals via stereotact injections, showing severe glioblastoma with hemorrhages.

FIGS. 5A-5E are H&E staining images of MSCs (FIG. 5A, arrow) found in the ventral part of the mid brain along the blood vessels (FIG. 5A, FIG. 5C) and maxillary sinus (FIG. 5B, arrow, FIG. 5D, FIG. 5E) 24 h after IN administration.

FIG. 6 showing H&E staining images of brain sections of GBM bearing mice at day 28, demonstrating a strong trend towards tumor reduction (lesion area) in IN treated ICOVIR5-infected mMSCs (Celyvir) mouse (bottom left) and in an ICOVIR5-infected MAV knockout mMSC IN treated mouse (bottom right), in comparison to vehicle (top left) and non-infected MSCs (top right).

FIG. 7 IBA-1 Immunohistochemistry staining images of brain sections of GBM bearing mice at day 28 post IN administration, showing intratumor lesion microglia infiltration trend (brown dots), in the brain of animals IN treated with ICOVIR5-infected mMSCs (Celyvir) (bottom images) in comparison to control animal (top images). ×4 (left) and ×10 (right) magnifications.

FIG. 8 IVIS scans of mice administered IN with vehicle (1M) or MSCs (2M/3M) labelled with PKH26 showing a specific localization in the brain area at 6, 24 and 48 hours post IN administration.

FIGS. 9A-9E PKH (FIG. 9A, 9C) and DAPI (FIGS. 9B, 9D) labelling of mouse brain tumor, of mouse IN treated with PKH26 labelled MSCs (Group 2M) (FIGS. 9A, 9B) and mouse IN treated with PKH26 labelled MAV knockout MSCs (Group 3M) (FIGS. 9C, 9D). Staining at 24 hours (FIGS. 9A, 9B) and 6 hours (FIGS. 9C, 9D) post IN administration. A clear labelling of positive cells (arrows) is shown in the tumor tissue. ×20 magnification. FIG. 9E—a semi-quantitative analysis of the histopathological findings, using a scoring scale. MSCs (2M/3M) labelled with PKH26 show a specific localization in the brain area throughout the study (2M). T-test showed a significant increase in luminescence score (P<0.01).

### DETAILED DESCRIPTION OF THE INVENTION

The present invention provides anti-cancer therapies. More specifically, the present invention provides compositions and methods, both prophylactic and therapeutic, for preventing and/or treating cancer.

Without being bound by any theory or mechanism, it is hypothesized that the combinatorial use of encapsulated oncolytic viruses (or virus-based vectors), and intranasal delivery, induce and/or promote the establishment, activity, and long-term potency of a clinically significant anti-cancer response in human patients.

The present invention provides, in one aspect, a method for treating cancer in a patient, the method comprising the step of administering to the patient a first active agent, the first active agent selected from the group consisting of an oncolytic virus and an oncolytic virus-based vector, wherein the first active agent: (i) is comprised in a cell, in an EV, or in a bioxome, and (ii) is administered intranasally to the patient.

The present invention provides, in another aspect, a method for treating cancer in a patient, the method comprising the step of administering to the patient a mesenchymal stem cell (MSC), an MSC-derived EV, or an MSC-derived bioxome. In certain embodiments, the method for treating cancer in a patient comprises the step of administering to the patient a mesenchymal stem cell (MSC). In certain embodiments, the method for treating cancer in a patient comprises the step of administering to the patient an MSC-derived EV. In certain embodiments, the method for treating cancer in a patient comprises the step of administering to the patient an MSC-derived bioxome.

In certain embodiments of the method for treating cancer, the MSC, MSC-derived EV, or MSC-derived bioxome comprises an oncolytic virus or an oncolytic virus-based vector. In certain embodiments of the method for treating cancer, the MSC comprises an oncolytic virus. In certain embodiments of the method for treating cancer, the MSC comprises an oncolytic virus-based vector. In certain embodiments of the method for treating cancer, the MSC-derived bioxome comprises an oncolytic virus. In certain embodiments of the method for treating cancer, the MSC-derived bioxome comprises an oncolytic virus-based vector. In certain embodiments of the method for treating cancer, the MSC-derived EV comprises an oncolytic virus. In certain embodiments of the method for treating cancer, the MSC-derived EV comprises an oncolytic virus-based vector.

In certain embodiments of the method for treating cancer, the MSC, MSC-derived EV, or MSC-derived bioxome is administered intranasally to the patient.

In certain embodiments, the cell is a mesenchymal stem cell (MSC). In certain embodiments, the EV is derived from a mesenchymal stem cell (MSC). In certain embodiments, the bioxome is derived from a mesenchymal stem cell (MSC).

The present invention provides, in another aspect, a method for treating cancer in a patient, the method comprising the step of administering to the patient a first active agent, the first active agent selected from the group consisting of an oncolytic virus and an oncolytic virus-based vector, wherein the first active agent: (i) is comprised in a mesenchymal stem cell (MSC), in an MSC-derived EV, or in an MSC-derived bioxome, and (ii) is administered intranasally to the patient.

A person of ordinary skill in the art would understand that the terminology of “first active agent”, “second active agent”, and “third active agent” is used merely to distinguish between active agents and does not necessarily indicate or relate to any particular order between the active agents or to any particular order of administering the active agents.

The present invention provides, in another aspect, a method for treating cancer in a patient, the method comprising the step of administering to the patient a pharmaceutical composition described herein.

### Administration.

In certain embodiments, the first active agent is administered intranasally to the brain of the patient.

In certain embodiments, the first active agent is applied to the olfactory nerve cells of the patient.

In certain embodiments, the first active agent is administered intranasally by a device selected from the group consisting of an instillation catheter, a dropper, a spray, a squeeze bottle, a pump spray, a compressed air nebulizer, a metered-dose inhaler, and an insufflator.

In certain embodiments, the first active agent is administered intranasally by an instillation catheter. In certain embodiments, the first active agent is administered intranasally by a dropper. In certain embodiments, the first active agent is administered intranasally by a spray. In certain embodiments, the first active agent is administered intranasally by a squeeze bottle. In certain embodiments, the first active agent is administered intranasally by a pump spray. In certain embodiments, the first active agent is administered intranasally by a compressed air nebulizer. In certain embodiments, the first active agent is administered intranasally by a metered-dose inhaler. In certain embodiments, the first active agent is administered intranasally by an insufflator. In certain embodiments, the first active agent is administered by a controlled particle dispersion device.

Kurve Technology has proprietary device called ViaNase™ (described in US Patents U.S. Pat. No. 7,231,919 B2 and U.S. Pat. No. 8,122,881, herein incorporated by reference in their entirety) that uses the principal of vortical flow, where Controlled Particle Dispersion (CPD)® effectively disrupts inherent nasal cavity airflows to deliver formulations to the entire nasal cavity and the brain. In certain embodiments, the first active agent is administered intranasally by the ViaNase™ device. In certain embodiments, the first active agent is administered intranasally by vortical flow device. In certain embodiments, the first active agent is administered intranasally by a Controlled Particle Dispersion device. In certain embodiments, the first active agent is administered intranasally by vortical flow, Controlled Particle Dispersion device. As used herein, the “N2B device” encompasses a nasal-to-brain device capable of intranasal administration.

In certain embodiments, the first active agent is administered intranasally by a controlled particle dispersion breathing method, the method performed by a user having a sinus, comprising: providing a nebulizer having a particle dispersion chamber to a user, the particle dispersion chamber capable of producing nebulized particles; activating the nebulizer; breathing a plurality of quick breaths as nebulized particles begin to flow out of the particle dispersion chamber; creating a pressure in the sinus of the user using the back of the throat; repeating the breathing of a plurality of quick breaths, holding the quick breaths and creating a pressure in the sinuses; breathing a plurality of long breaths; and repeating the breathing of a plurality of quick breaths, holding the quick breaths, creating a pressure in the sinuses and breathing a plurality of long breaths.

In certain embodiments, the first active agent is administered intranasally by a particle dispersion device for nasal delivery, comprising: a nasal adapter having an input and an output; a particle dispersion chamber having an input opening and an output opening, the output opening in communication with the nasal adapter input, the chamber having directed fluid outputs operative to impart a vortical flow to aerosolized particles exiting the chamber output opening and entering the nasal adapter; an outflow tube in communication with the input opening of the particle dispersion chamber; and a housing, the housing having a medicine chamber in which a medicine is nebulized or aerosolized, in communication with the outflow tube.

In certain embodiments, the first active agent is administered intranasally by a method of delivering a medicament to the nasal cavity and paranasal sinuses, comprising: providing a particle dispersion device, comprising: a nasal adapter having an input and an output; a particle dispersion chamber having an input opening and an output opening, the output opening in communication with the nasal adapter input, the chamber having fluid outputs operative to impart a vortical flow to aerosolized particles exiting the chamber output opening and entering the nasal adapter; an outflow tube in communication with the input opening of particle dispersion chamber; and a housing, the housing having a medicine chamber in which a medicine is nebulized or aerosolized, in communication with the outflow tube; performing the controlled particle dispersion breathing technique described above; and delivering the medicament.

In certain embodiments, the first active agent is administered intranasally by a particle dispersion chamber, comprising: a nasal adapter having an input and an output; a housing having an external surface, an input opening, an output opening, and an internal channel between the openings, the channel generally defining an axis and a forward direction toward the output opening, the output opening in communication with the nasal adapter input; and a plurality of directional fluid outputs within and communicating with the internal channel, directing output at an acute forward angle with respect to the channel axis and output opening, wherein the fluid outputs, when fluid flows therefrom, are operative to impart a vortical flow to aerosolized particles exiting the output opening and entering the nasal adapter, and wherein the internal channel has a substantially continuous cross-sectional area from and including a location of a fluid output within the internal channel to, and including, the output opening.

In certain embodiments, the first active agent is administered intranasally by a particle dispersion chamber, comprising: a nasal adapter having an input and an output; a housing having an external surface, an input opening, an output opening, and an internal channel between the openings, the channel generally defining an axis and a forward direction toward the output opening, the output opening in communication with the nasal adapter input; and a plurality of directional fluid outputs within and communicating with the internal channel, directing output at an acute forward angle with respect to the channel axis and output opening, wherein the fluid outputs, when fluid flows therefrom, are operative to impart a vortical flow to aerosolized particles exiting the output opening and entering the nasal adapter; and wherein the internal channel has a substantially constant cross-sectional area from and including a localion a fluid output within the internal channel to, and including, the output opening.

In certain embodiments, the first active agent is administered intranasally by a particle dispersion device for nasal delivery suitable for topical drug delivery to, or systemic drug delivery via, a deep nasal cavity or paranasal sinus, comprising: a nasal adapter having an input and an output; a particle dispersion chamber having a particle entry end and an output opening and an internal channel between the particle input end and the output opening, the output opening in communication with the nasal adapter input, the particle dispersion chamber having directed fluid outputs within and communicating with the internal channel and configured to direct output forward to impart a vortical flow to aerosolized particles exiting the dispersion chamber output opening and entering the nasal adapter, and wherein the internal channel has a substantially continuous cross-sectional area from the particle input end of the housing and including a location of a fluid output within the internal channel to, and including, the output opening; a nebulizing or aerosolizing chamber in direct communication with the particle dispersion chamber, and in which a medicine is nebulizable or aerosolizable; and a nebulizing or aerosolizing pressure feed in communication with the nebulizing or aerosolizing chamber, and having a compressor channel to channel compressed fluid.

In certain embodiments, the first active agent is administered intranasally by a method for topical drug delivery to, or systemic drug delivery via, a deep nasal cavity or paranasal sinus, comprising: providing the particle dispersion device for nasal delivery described above; and breathing medicament-containing particles therefrom through a nasal cavity, wherein topical drug delivery to, or systemic drug delivery via, a deep nasal cavity or paranasal sinus is afforded.

### Cancers

In one embodiment, disclosed herein are methods for treating, preventing, inhibiting, reducing the incidence of, ameliorating, or alleviating a cancer or a tumor comprising the step of administering a first active agent selected from the group consisting of an oncolytic virus and an oncolytic virus-based vector, as described herein, wherein the first active agent: (i) is comprised in a cell, in an EV, or in a bioxome, and (ii) is administered intranasally to the patient.

In some embodiments, disclosed herein are methods of treating, preventing, inhibiting, reducing the incidence of, ameliorating, or alleviating a cancer or a tumor in a subject comprising the step of administering any of the active agents described herein. In some embodiments, disclosed herein are methods of treating, preventing, inhibiting the growth of, delaying disease progression, reducing the tumor load, or reducing the incidence of a cancer or a tumor in a subject, or any combination thereof. In some embodiments, methods disclosed herein reduce the size and or growth rate of a tumor or cancer. In some embodiments, methods disclosed herein increase the survival of a subject suffering from a tumor or cancer.

In some embodiments the method for treating cancer in a patient, the method comprises the step of administering to the patient a mesenchymal stem cell (MSC), an MSC-derived EV, or an MSC-derived bioxome. In certain embodiments, the method for treating cancer in a patient comprises the step of administering to the patient a mesenchymal stem cell (MSC). In certain embodiments, the method for treating cancer in a patient comprises the step of administering to the patient an MSC-derived EV. In certain embodiments, the method for treating cancer in a patient comprises the step of administering to the patient an MSC-derived bioxome.

In certain embodiments of the method for treating cancer, the MSC, MSC-derived EV, or MSC-derived bioxome comprise an oncolytic virus or an oncolytic virus-based vector. In certain embodiments of the method for treating cancer, the MSC comprises an oncolytic virus. In certain embodiments of the method for treating cancer, the MSC comprises an oncolytic virus-based vector. In certain embodiments of the method for treating cancer, the MSC-derived bioxome comprises an oncolytic virus. In certain embodiments of the method for treating cancer, the MSC-derived bioxome comprises an oncolytic virus-based vector. In certain embodiments of the method for treating cancer, the MSC-derived EV comprises an oncolytic virus. In certain embodiments of the method for treating cancer, the MSC-derived EV comprises an oncolytic virus-based vector.

In certain embodiments of the method for treating cancer, the MSC, MSC-derived EV, or MSC-derived bioxome is administered intranasally to the patient.

In certain embodiments, disclosed herein is a method of treating a cancer or a tumor in a subject, said method comprising the step of administering to a subject any of the active agents described herein. In another embodiment, disclosed herein is a method of preventing a cancer or a tumor in a subject, said method comprising the step of administering to said subject any of the active agents described herein. In another embodiment, disclosed herein is a method of inhibiting a cancer or a tumor in a subject, said method comprising the step of administering to said subject any of the active agents described herein. In another embodiment, disclosed herein is a method of reducing a cancer or a tumor in a subject, said method comprising the step of administering to said subject any of the active agents described herein. In another embodiment, disclosed herein is a method of ameliorating a cancer or a tumor in a subject, said method comprising the step of administering to said subject any of the active agents described herein. In another embodiment, disclosed herein is a method of alleviating a cancer or a tumor in a subject, said method comprising the step of administering to said subject any of the active agents described herein.

In some embodiment, “treating” comprises therapeutic treatment and “preventing” comprises prophylactic or preventative measures, wherein the object is to prevent or lessen the targeted pathologic condition or disorder as described hereinabove. Thus, in some embodiments, treating may include directly affecting or curing, suppressing, inhibiting, preventing, reducing the severity of, delaying the onset of, reducing symptoms associated with the disease, disorder or condition, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In certain embodiments, the cancer is CNS cancer. In certain embodiments, the cancer is brain cancer. In certain embodiments, the brain cancer is glioblastoma. In certain embodiments, the brain cancer is glioblastoma multiforme. In certain embodiments, the brain cancer is glioma. In certain embodiments, the cancer is a solid tumor. In certain embodiments, the cancer is a primary tumor. In certain embodiments, the cancer is a metastatic tumor. In certain embodiments, the solid tumor is relapsed/refractory solid tumor.

### Oncolytic Viruses and Oncolytic Virus-Based Vectors

In certain embodiments, the first active agent is an oncolytic virus.

The term “oncolytic virus”, as used herein, generally refers to a virus that (i) preferentially infects cancer cells, (ii) preferentially replicates in cancer cells, and/or (iii) preferentially kills cancer cells.

In certain embodiments, the oncolytic virus infects and kills cancer cells.

In certain embodiments, the oncolytic virus is selected from the group consisting of a Herpes simplex virus (HSV), an Adenovirus, a Vaccinia virus (VACV), a Vesicular stomatitis virus (VSV), a Poliovirus, a Reovirus, a Senecavirus, an Echovirus, a Semliki Forest virus (SFV), a Maraba virus, and an Enterovirus.

In certain embodiments, the oncolytic virus is an HSV. In certain embodiments, the oncolytic virus is an Adenovirus. In certain embodiments, the oncolytic virus is a VACV. In certain embodiments, the oncolytic virus is a VSV. In certain embodiments, the oncolytic virus is a Poliovirus. In certain embodiments, the oncolytic virus is a Reovirus. In certain embodiments, the oncolytic virus is a Senecavirus. In certain embodiments, the oncolytic virus is an Echovirus. In certain embodiments, the oncolytic virus is a SFV. In certain embodiments, the oncolytic virus is a Maraba virus. In certain embodiments, the oncolytic virus is Enterovirus.

In certain embodiments, the oncolytic virus is first active agent is an oncolytic virus-based vector.

The term “oncolytic virus-based vector”, as used herein, generally refers to a virus-based vector that (i) preferentially infects cancer cells, (ii) preferentially replicates in cancer cells, and/or (iii) preferentially kills cancer cells.

In certain embodiments, the virus-based vector infects and kills cancer cells.

A person of ordinary skill in the art would understand that the terminology of “virus-based vector” relates to any vector which is structurally and/or genetically similar to a known virus. In certain embodiments, the virus-based vector is at least 50% structurally and/or genetically identical to a known virus. In certain embodiments, the virus-based vector is at least 75% structurally and/or genetically identical to a known virus. In certain embodiments, the virus-based vector is at least 90% structurally and/or genetically identical to a known virus. In certain embodiments, the virus-based vector is at least 95% structurally and/or genetically identical to a known virus. In certain embodiments, the virus-based vector is at least 99% structurally and/or genetically identical to a known virus.

In certain embodiments, the oncolytic virus-based vector is selected from the group consisting of an Adenovirus-based vector, an HSV-based vector, a VACV-based vector, a VSV-based vector, a Poliovirus-based vector, a Reovirus-based vector, a Senecavirus-based vector, an Echovirus-based vector, a SFV-based vector, a Maraba virus-based vector, and an Enterovirus-based vector.

In certain embodiments, the oncolytic virus-based vector is an Adenovirus-based vector. In certain embodiments, the oncolytic virus-based vector is an HSV-based vector. In certain embodiments, the oncolytic virus-based vector is a VACV-based vector. In certain embodiments, the oncolytic virus-based vector is a VSV-based vector. In certain embodiments, the oncolytic virus-based vector is a Poliovirus-based vector. In certain embodiments, the oncolytic virus-based vector is a Reovirus-based vector. In certain embodiments, the oncolytic virus-based vector is a Senecavirus-based vector. In certain embodiments, the oncolytic virus-based vector is an Echovirus-based vector. In certain embodiments, the oncolytic virus-based vector is a SFV-based vector. In certain embodiments, the oncolytic virus-based vector is a Maraba virus-based vector. In certain embodiments, the oncolytic virus-based vector is an Enterovirus-based vector.

In certain embodiments, the oncolytic virus-based vector is selected from the group consisting of ICOVIR-5, a Herpes simplex virus type 1 mutant 1716 (HSV-1716), Oncorine (H101), Onyx-15 (dl1520), ColoAd1, Talimogene laherparepvec (T-VEC), GL-ONC1, CV706, and GLV-1h68.

In certain embodiments, the oncolytic virus-based vector is ICOVIR-5. In certain embodiments, the oncolytic virus-based vector is a HSV-1716. In certain embodiments, the oncolytic virus-based vector is H101. In certain embodiments, the oncolytic virus-based vector is Onyx-15. In certain embodiments, the oncolytic virus-based vector is ColoAd1. In certain embodiments, the oncolytic virus-based vector is T-VEC. In certain embodiments, the oncolytic virus-based vector is GL-ONC1. In certain embodiments, the oncolytic virus-based vector is CV706. In certain embodiments, the oncolytic virus-based vector is GLV-1h68.

ICOVIR-5 (Ad-DM-E2F-K-Δ24RGD) is an AdΔ24RGD-derivative oncolytic adenovirus that has been designed to increase replication potency in tumor cells compared with ICOVIR-2 (Mol Ther. 2007 September; 15 (9): 1607-15, incorporated herein by reference).

In certain embodiments, the first active agent is comprised in an MSC. In certain embodiments, the first active agent is comprised in an MSC-derived EV. In certain embodiments, the first active agent is comprised in an MSC-derived bioxome.

### Mesenchymal Stem Cells (MSCs)

International PCT Application Publication WO 2019/043282, and Spanish Patent ES 2702618 B2, relating, inter-alia, to cell-encapsulated oncolytic viruses, such as “Celyvir”, are herein incorporated by reference in their entirety. In some embodiments, mesenchymal stem cells (MSCs) carrying the oncolytic adenovirus ICOVIR-5, are called Celyvir.

In some embodiments, the term “mesenchymal stem cell” (“MSC”) may encompass multipotent progenitor cells derived from the mesoderm. In some embodiments, MSCs can differentiate into cells that make up adipose, bone, cartilage and muscle tissue. In some embodiments, MSCs are positive for CD70, CD90 and CD105, and negatives for CD45 and CD34. In some embodiments, the MSCs described herein are derived from bone marrow, peripheral blood, menstrual blood, salivary gland, skin and foreskin, synovial fluid, endometrium, dental tissue, adipose tissue and tissues associated with newborns including placenta, umbilical cord, cord blood umbilical, amniotic fluid and amniotic membrane.

In certain embodiments, in the MSC, the expression and activity of different genes and proteins is not artificially inhibited in order to decrease the immunogenicity of the MSC.

In certain embodiments, in the MSC, (i) a Toll-like receptor selected from the group consisting of TLR1, TLR2, TLR4, TLR5, TLR6, TLR7, TLR8, TLR9, and TLR10, gene or protein, is not inhibited, (ii) the MyD88 gene or protein is not inhibited, or (iii) the MAVS gene or protein is not inhibited.

In certain embodiments, in the MSC, (i) a Toll-like receptor selected from the group consisting of TLR1, TLR2, TLR4, TLR5, TLR6, TLR7, TLR8, TLR9, and TLR10, gene or protein, is not inhibited, (ii) the MyD88 gene or protein is not inhibited, and (iii) the MAVS gene or protein is not inhibited.

In certain embodiments, in the MSC, the expression and activity of different genes and proteins is artificially inhibited in order to decrease the immunogenicity of the MSC.

In certain embodiments, in the MSC, (i) a Toll-like receptor selected from the group consisting of TLR1, TLR2, TLR4, TLR5, TLR6, TLR7, TLR8, TLR9, and TLR10, gene or protein, is inhibited, (ii) the MyD88 gene or protein is inhibited, or (iii) the MAVS gene or protein is inhibited.

In certain embodiments, in the MSC, (i) a Toll-like receptor selected from the group consisting of TLR1, TLR2, TLR4, TLR5, TLR6, TLR7, TLR8, TLR9, and TLR10, gene or protein, is inhibited, (ii) the MyD88 gene or protein is inhibited, and (iii) the MAVS gene or protein is inhibited.

In certain embodiments, in the MSC, TLR4, TLR9, or the MyD88, gene or protein, is inhibited. In certain embodiments, in the MSC, TLR4, TLR9, and the MyD88, gene or protein, are inhibited.

In certain embodiments, in the MSC, the MAVS gene or protein is inhibited.

In certain embodiments, the mesenchymal stem cell is a human mesenchymal stem cell. In certain embodiments, the mesenchymal stem cell is a human cancer patient mesenchymal stem cell. In certain embodiments, the mesenchymal stem cell is autologous to a human cancer patient.

In certain embodiments, the mesenchymal stem cell is derived from a source selected from the group consisting of bone marrow, placenta, umbilical cord, amniotic membrane, menstrual blood, peripheral blood, salivary gland, skin, foreskin, synovial fluid, amniotic fluid, endometrium, adipose tissue, cord blood, and dental tissue. In certain embodiments, the mesenchymal stem cell is derived from bone marrow (BM-MSC).

In certain embodiments, the expression of the MAVS gene, or of other genes regulated by the MAVS gene, in the mesenchymal stem cell, is inhibited. In certain embodiments, the expression of the MAVS gene in the mesenchymal stem cell is inhibited. In certain embodiments, the expression of a gene regulated by the MAVS gene in the mesenchymal stem cell is inhibited.

In certain embodiments, the gene regulated by the MAVS gene is selected from the group consisting of NF-Kappa-B, IRF3, and IRF7.

### Extracellular Vesicles

The term “extracellular vesicles (EVs)”, as used herein, refers to membrane-derived microvesicles, which includes a range of extracellular vesicles, including exosomes, microparticles, shed microvesicles, oncosomes, ectosomes, secreted by many cell types under both normal physiological and pathological conditions. The term “exosome” may encompass intracellular vesicles, plant secretome vesicles, microbiome vesicles, and retroviral-like particles of all sizes.

### Bioxomes

International PCT Application Publication WO 2019/198068, to the present applicant, describes artificial bioxome particles, and methods for their production, and is herein incorporated by reference.

In certain embodiments, the artificial bioxome particle: comprises a cell membrane component derived from a cellular or extracellular source; is designed to undergo fusion with a target cell; is engineered to carry a cargo comprising at least one predetermined active molecule, wherein the cargo can be released into a target cell after the fusion of the bioxome particle with the target cell; and any combination thereof.

In certain embodiments, the artificial bioxome particle comprises a cell membrane component and designed to undergo fusion with a target cell, wherein the bioxome particle is engineered to carry a cargo comprising at least one predetermined active molecule; and wherein the cargo can be released into the target cell after the fusion of the bioxome particle with the target cell; and wherein the cell membrane component is derived from a selected cellular or extracellular source.

In certain embodiments, the cargo comprises at least two active molecules.

In certain embodiments, the cargo comprises a plurality of active molecules.

In certain embodiments, the source is selected from the group consisting of fibroblasts, mesenchymal stem cells, stem cells, cells of the immune system, dendritic cells, ectoderm, keratinocytes, cells of GI, cells of oral cavity, nasal mucosal cells, neuronal cells, retinal cells, endothelial cells, cardiospheres, cardiomyocytes, pericytes, blood cells, melanocytes, parenchymal cells, liver reserve cells, neural stem cells, pancreatic stem cells, embryonic stem cells, bone marrow, skin tissue, liver tissue, pancreatic tissue, biological fluids, excrement or surgery extracted tissues, milk, saliva, mucus, blood plasma, urine, feces, sebum, postnatal umbilical cord, placenta, amniotic sac, kidney tissue, neurological tissue, adrenal gland tissue, mucosal epithelium, smooth muscle tissue, a bacterial cell, a bacterial culture, a whole microorganism, conditional medium, amniotic fluid, lipoaspirate, liposuction byproducts, and a plant tissue.

In certain embodiments, the source is fibroblasts. In certain embodiments, the source is mesenchymal stem cells. In certain embodiments, the source is stem cells. In certain embodiments, the source is cells of the immune system. In certain embodiments, the source is dendritic cells. In certain embodiments, the source is ectoderm. In certain embodiments, the source is keratinocytes. In certain embodiments, the source is cells of GI. In certain embodiments, the source is cells of oral cavity. In certain embodiments, the source is nasal mucosal cells. In certain embodiments, the source is neuronal cells. In certain embodiments, the source is retinal cells. In certain embodiments, the source is endothelial cells. In certain embodiments, the source is cardiospheres. In certain embodiments, the source is cardiomyocytes. In certain embodiments, the source is pericytes. In certain embodiments, the source is blood cells. In certain embodiments, the source is melanocytes. In certain embodiments, the source is parenchymal cells. In certain embodiments, the source is liver reserve cells. In certain embodiments, the source is neural stem cells. In certain embodiments, the source is pancreatic stem cells. In certain embodiments, the source is embryonic stem cells. In certain embodiments, the source is bone marrow. In certain embodiments, the source is skin tissue. In certain embodiments, the source is liver tissue. In certain embodiments, the source is pancreatic tissue. In certain embodiments, the source is biological fluids. In certain embodiments, the source is excrement or surgery extracted tissues. In certain embodiments, the source is milk. In certain embodiments, the source is saliva. In certain embodiments, the source is mucus. In certain embodiments, the source is blood plasma. In certain embodiments, the source is urine. In certain embodiments, the source is feces. In certain embodiments, the source is sebum. In certain embodiments, the source is postnatal umbilical cord. In certain embodiments, the source is placenta. In certain embodiments, the source is amniotic sac. In certain embodiments, the source is kidney tissue. In certain embodiments, the source is neurological tissue. In certain embodiments, the source is adrenal gland tissue. In certain embodiments, the source is mucosal epithelium. In certain embodiments, the source is smooth muscle tissue. In certain embodiments, the source is a bacterial cell. In certain embodiments, the source is a bacterial culture. In certain embodiments, the source is a whole microorganism. In certain embodiments, the source is conditional medium. In certain embodiments, the source is amniotic fluid. In certain embodiments, the source is lipoaspirate. In certain embodiments, the source is liposuction byproducts. In certain embodiments, the source is a plant tissue.

In certain embodiments, the source is mesenchymal stem cells. In certain embodiments, the source is normal mesenchymal stem cells. In certain embodiments, the source is mutated mesenchymal stem cells. In certain embodiments, the source is mutated mesenchymal stem cells, comprising a mutation in a gene selected from the group consisting of TLR1, TLR2, TLR4, TLR5, TLR6, TLR7, TLR8, TLR9, and TLR10, and MyD88. In certain embodiments, the source is mutated mesenchymal stem cells, comprising a mutation in a MAVS gene.

In certain embodiments, the active molecule is selected from the group consisting of nucleic acid, peptide, amino acid, polypeptide, nucleoside, growth factor, organic molecule, polyphenol, steroid, lipophilic poor soluble drug, inorganic molecule, anti-oxidant, hormone, antibody, vitamin, cytokine, enzyme, heat shock protein, or a combination thereof.

In certain embodiments, the active molecule is nucleic acid. In certain embodiments, the source is peptide. In certain embodiments, the source is amino acid. In certain embodiments, the source is polypeptide. In certain embodiments, the source is nucleoside. In certain embodiments, the source is growth factor. In certain embodiments, the source is organic molecule. In certain embodiments, the source is polyphenol. In certain embodiments, the source is steroid. In certain embodiments, the source is lipophilic poor soluble drug. In certain embodiments, the source is inorganic molecule. In certain embodiments, the source is anti-oxidant. In certain embodiments, the source is hormone. In certain embodiments, the source is antibody. In certain embodiments, the source is vitamin. In certain embodiments, the source is cytokine. In certain embodiments, the source is enzyme. In certain embodiments, the source is heat shock protein. In certain embodiments, the source is a combination thereof.

In certain embodiments, the active molecule is selected from the group consisting of cannabinoid, cannabinoid acid, and endocannabinoid.

In certain embodiments, the nucleic acid is RNA, and is selected from the group consisting of siRNA, an antisense RNA, iRNA, microRNA, an antagomir, an aptamer, and a ribozyme mRNA.

In certain embodiments, the nucleic acid is siRNA. In certain embodiments, the nucleic acid is an antisense RNA. In certain embodiments, the nucleic acid is iRNA. In certain embodiments, the nucleic acid is microRNA. In certain embodiments, the nucleic acid is an antagomir. In certain embodiments, the nucleic acid is an aptamer. In certain embodiments, the nucleic acid is a ribozyme mRNA.

In certain embodiments, the active molecule has a therapeutic effect.

In certain embodiments, the therapeutic effect is selected from the group consisting of anti-fibrotic effect, anti-tumor effect, and neuroprotective effect.

In certain embodiments, the therapeutic effect is anti-fibrotic effect. In certain embodiments, the therapeutic effect is anti-tumor effect. In certain embodiments, the therapeutic effect is neuroprotective effect. In certain embodiments, the therapeutic effect is an anti-tumor effect and a neuroprotective effect.

In certain embodiments, the bioxome is a redoxome, wherein the cargo comprises at least one redox active free-radicals scavenging compound.

In certain embodiments, the cargo comprises at least one redox active free-radicals scavenging compound.

In certain embodiments, the bioxome comprises fenton reaction complex blockers, hydroxyl radical trap, iron chelator and a lipid radical trap.

In certain embodiments, the bioxome is capable of blocking LPO chain reaction, such as lipid radical/peroxide trap, such as vitamin E, terpenoids, polyphenols, flavonoid, phenolic acids, cannabinoids, retinoids, vitamin D, lipoic acid, sterols.

In certain embodiments, the radical trap is ascorbic acid, nitric oxid donor (S-nitrosoglutathione), or a derivative thereof.

In certain embodiments, the iron chelator is selected from the group consisting of des ferrioxamine (DFX), ethylenediaminetetraacetic acid (EDTA), rutin, disodium EDTA, tetrasodium EDTA, calcium disodium EDTA, diethylenetriaminepentaacetic acid (DTPA) or a salt thereof, hydroxyethlethylenediaminetriacetic acid (HEDTA) or a salt thereof, nitrilotriacetic acid (NTA), acetyl trihexyl citrate, aminotrimethylene phosphonic acid, beta-alanine diacetic acid, bismuth citrate, citric acid, cyclohexanediamine tetraacetic acid, diammonium citrate, dibutyl oxalate, diethyl oxalate, diisobutyl oxalate, diisopropyl oxalate, dilithium oxalate, dimethyl oxalate, dipotassium EDTA, dipotassium oxalate, dipropyl oxalate, disodium EDTA-copper, disodium pyrophosphate, etidronic acid, HEDTA, methyl cyclodextrin, oxalic acid, pentapotassium, triphosphate, pentasodium aminotrimethylene phosphonate, pentasodium pentetate, pentasodium triphosphate, pentetic acid, dicarboxyic acid, phytic acid, potassium citrate, sodium citrate, sodium dihydroxyethylglycinate, sodium gluceptate, sodium gluconate, sodium hexametaphosphate, sodium metaphosphate, sodium metasilicate, sodium oxalate, sodium trimetaphosphate, tea-EDTA, tetrahydroxypropyl ethylenediamine, tetrapotassium etidronate, tetrapotassium pyrophosphate, tetrasodium etidronate, tetrasodium pyrophosphate, tripotassium EDTA, trisodium EDTA, trisodium hedta, trisodium NTA, trisodium phosphate, malic acid, fumaric acid, maltol, succimer, penicillamine, dimercaprol, deferipron, a natural protein based iron chelator, melatonin, siderphore, zinc or copper cation, or salt or complex, and desferrioxamine mesylate, or a combination thereof.

In certain embodiments, the iron chelator is des ferrioxamine (DFX). In certain embodiments, the iron chelator is ethylenediaminetetraacetic acid (EDTA). In certain embodiments, the iron chelator is rutin. In certain embodiments, the iron chelator is disodium EDTA. In certain embodiments, the iron chelator is tetrasodium EDTA. In certain embodiments, the iron chelator is calcium disodium EDTA. In certain embodiments, the iron chelator is diethylenetriaminepentaacetic acid (DTPA) or a salt thereof. In certain embodiments, the iron chelator is hydroxyethlethylenediaminetriacetic acid (HEDTA) or a salt thereof. In certain embodiments, the iron chelator is nitrilotriacetic acid (NTA). In certain embodiments, the iron chelator is acetyl trihexyl citrate. In certain embodiments, the iron chelator is aminotrimethylene phosphonic acid. In certain embodiments, the iron chelator is beta-alanine diacetic acid. In certain embodiments, the iron chelator is bismuth citrate. In certain embodiments, the iron chelator is citric acid. In certain embodiments, the iron chelator is cyclohexanediamine tetraacetic acid. In certain embodiments, the iron chelator is diammonium citrate. In certain embodiments, the iron chelator is dibutyl oxalate. In certain embodiments, the iron chelator is diethyl oxalate. In certain embodiments, the iron chelator is diisobutyl oxalate. In certain embodiments, the iron chelator is diisopropyl oxalate. In certain embodiments, the iron chelator is dilithium oxalate. In certain embodiments, the iron chelator is dimethyl oxalate. In certain embodiments, the iron chelator is dipotassium EDTA. In certain embodiments, the iron chelator is dipotassium oxalate. In certain embodiments, the iron chelator is dipropyl oxalate. In certain embodiments, the iron chelator is disodium EDTA-copper. In certain embodiments, the iron chelator is disodium pyrophosphate. In certain embodiments, the iron chelator is etidronic acid. In certain embodiments, the iron chelator is HEDTA. In certain embodiments, the iron chelator is methyl cyclodextrin. In certain embodiments, the iron chelator is oxalic acid. In certain embodiments, the iron chelator is pentapotassium. In certain embodiments, the iron chelator is triphosphate. In certain embodiments, the iron chelator is pentasodium aminotrimethylene phosphonate. In certain embodiments, the iron chelator is pentasodium pentetate. In certain embodiments, the iron chelator is pentasodium triphosphate. In certain embodiments, the iron chelator is pentetic acid. In certain embodiments, the iron chelator is dicarboxyic acid. In certain embodiments, the iron chelator is phytic acid. In certain embodiments, the iron chelator is potassium citrate. In certain embodiments, the iron chelator is sodium citrate. In certain embodiments, the iron chelator is sodium dihydroxyethylglycinate. In certain embodiments, the iron chelator is sodium gluceptate. In certain embodiments, the iron chelator is sodium gluconate. In certain embodiments, the iron chelator is sodium hexametaphosphate. In certain embodiments, the iron chelator is sodium metaphosphate. In certain embodiments, the iron chelator is sodium metasilicate. In certain embodiments, the iron chelator is sodium oxalate. In certain embodiments, the iron chelator is sodium trimetaphosphate. In certain embodiments, the iron chelator is tea-EDTA. In certain embodiments, the iron chelator is tetrahydroxypropyl ethylenediamine. In certain embodiments, the iron chelator is tetrapotassium etidronate. In certain embodiments, the iron chelator is tetrapotassium pyrophosphate. In certain embodiments, the iron chelator is tetrasodium etidronate. In certain embodiments, the iron chelator is tetrasodium pyrophosphate. In certain embodiments, the iron chelator is tripotassium EDTA. In certain embodiments, the iron chelator is trisodium EDTA. In certain embodiments, the iron chelator is trisodium hedta. In certain embodiments, the iron chelator is trisodium NTA. In certain embodiments, the iron chelator is trisodium phosphate. In certain embodiments, the iron chelator is malic acid. In certain embodiments, the iron chelator is fumaric acid. In certain embodiments, the iron chelator is maltol. In certain embodiments, the iron chelator is succimer. In certain embodiments, the iron chelator is penicillamine. In certain embodiments, the iron chelator is dimercaprol. In certain embodiments, the iron chelator is deferipron. In certain embodiments, the iron chelator is a natural protein based iron chelator. In certain embodiments, the iron chelator is melatonin. In certain embodiments, the iron chelator is siderphore. In certain embodiments, the iron chelator is zinc or copper cation or salt or complex. In certain embodiments, the iron chelator is desferrioxamine mesylate.

In certain embodiments, the iron chelator is selected from the group consisting of EDTA (ethylenediaminetetraacetic acid), DTPA (diethylene triamine pentaacetic acid), NTA (nitrilotriacetic acid), detoxamin, deferoxamine, defferiprone, deferasirox, glutathione, metalloprotein, ferrochel (bis-glycinate chelate), ceruloplasmin, penicillamine, cuprizone, trientine, ferrulic acid, zinc acetate, lipocalin 2, and dimercaprol.

In certain embodiments, the iron chelator is EDTA (ethylenediaminetetraacetic acid). In certain embodiments, the iron chelator is DTPA (diethylene triamine pentaacetic acid). In certain embodiments, the iron chelator is NTA (nitrilotriacetic acid). In certain embodiments, the iron chelator is detoxamin. In certain embodiments, the iron chelator is deferoxamine. In certain embodiments, the iron chelator is deferiprone. In certain embodiments, the iron chelator is deferasirox. In certain embodiments, the iron chelator is glutathione. In certain embodiments, the iron chelator is metalloprotein. In certain embodiments, the iron chelator is ferrochel (bis-glycinate chelate). In certain embodiments, the iron chelator is ceruloplasmin. In certain embodiments, the iron chelator is penicillamine. In certain embodiments, the iron chelator is cuprizone. In certain embodiments, the iron chelator is trientine. In certain embodiments, the iron chelator is ferrulic acid. In certain embodiments, the iron chelator is zinc acetate. In certain embodiments, the iron chelator is lipocalin 2. In certain embodiments, the iron chelator is dimercaprol.

In certain embodiments, the bioxome is a long circulating, slow release bioxome.

In certain embodiments, the bioxome is a selective targeting bioxome.

In certain embodiments, the bioxome is an immunogenic bioxome.

### MAVS-Inhibited MSCs

The present invention provides, in another aspect, a mesenchymal stem cell (MSC), wherein in the MSC, the MAVS gene or protein is inhibited.

The present invention provides, in a related aspect, an EV derived from the MAVS-inhibited MSC.

The present invention provides, in a related aspect, a bioxome derived from the MAVS-inhibited MSC.

The present invention provides, in another aspect, a mesenchymal stem cell (MSC), wherein in the MSC, a gene or protein regulated by the MAVS gene is inhibited.

The present invention provides, in a related aspect, an EV derived from the MSC in which a gene or protein regulated by the MAVS gene is inhibited.

The present invention provides, in a related aspect, a bioxome derived from the MSC in which a gene or protein regulated by the MAVS gene is inhibited.

The present invention provides, in another aspect, a method for the production of a substantially pure population of modified mesenchymal stem cells, the method comprising the steps of: (i) obtaining a sample of mesenchymal stem cells, (ii) culturing the mesenchymal stem cells obtained in step (i), and (iii) inhibiting the MAVS gene or protein, or of a gene or protein regulated by the MAVS gene or protein, in the cells cultured in step (ii).

### Compositions

Also embraced within this invention are pharmaceutical compositions comprising the first active agent described herein in detail. In some embodiments, the pharmaceutical composition comprises a mesenchymal stem cell (MSC) comprising the first active agent described herein. In some embodiments, the pharmaceutical composition comprises an MSC-derived EV comprising the first active agent described herein. In some embodiments, the pharmaceutical composition comprises an MSC-derived bioxome comprising the first active agent described herein.

In certain embodiments the MSC, MSC-derived EV, or MSC-derived bioxome comprises an oncolytic virus or an oncolytic virus-based vector. In certain embodiments, the MSC comprises an oncolytic virus. In certain embodiments, the MSC comprises an oncolytic virus-based vector. In certain embodiments, the MSC-derived bioxome comprises an oncolytic virus. In certain embodiments, the MSC-derived bioxome comprises an oncolytic virus-based vector. In certain embodiments, the MSC-derived EV comprises an oncolytic virus. In certain embodiments, the MSC-derived EV comprises an oncolytic virus-based vector.

In certain embodiments, the oncolytic virus-based vector is selected from the group consisting of an Adenovirus-based vector, an HSV-based vector, a VACV-based vector, a VSV-based vector, a Poliovirus-based vector, a Reovirus-based vector, a Senecavirus-based vector, an Echovirus-based vector, a SFV-based vector, a Maraba virus-based vector, and an Enterovirus-based vector.

In certain embodiments, the oncolytic virus-based vector is selected from the group consisting of ICOVIR-5 (Ad-DM-E2F-K-Δ24RGD), a Herpes simplex virus type 1 mutant 1716 (HSV-1716), Oncorine (H101), Onyx-15 (dl1520), ColoAd1, Talimogene laherparepvec (T-VEC), GL-ONC1, CV706, and GLV-1h68. In certain embodiments, the oncolytic virus-based vector is ICOVIR-5.

In some embodiments, the pharmaceutical is administered intranasally to the patient.

In some embodiments, the pharmaceutical further comprises pharmaceutically acceptable carriers and/or diluents and/or adjuvants (collectively referred to herein as “carrier” materials) and, if desired, other active ingredients.

The pharmaceutical compositions of this invention can be processed in accordance with conventional methods of pharmacy to produce medicinal compositions for administration to patients, including humans and other mammals. The pharmaceutical compositions may be subjected to conventional pharmaceutical operations and/or may contain conventional adjuvants, buffers etc.

The amounts of the pharmaceutical composition to be administered and the dosage regimen for treating a disease condition with compositions of this invention depends on a variety of factors, including the age, weight, gender, the medical condition of the subject, the type of disease, the severity of the disease, and the route and frequency of administration. Thus, the dosage regimen may vary widely, but can be determined routinely using standard methods.

### Central Nervous System Related Disorders

In certain embodiments, the present invention provides central nervous system (CNS)-related disorder therapies.

In certain embodiments, the present invention relates to a method for treating a central nervous system (CNS)-related disorder, the method comprising administering to the subject a mesenchymal stem cell (MSC), an MSC-derived EV, or an MSC-derived bioxome. In certain embodiments, the administration is intranasal administration to the subject.

In certain embodiments, the present invention relates to a method for treating central nervous system (CNS) related disorders, the method comprising administering to the subject a mesenchymal stem cell (MSC), an MSC-derived EV, or an MSC-derived bioxome, wherein said administration is intranasal administration to the subject.

In certain embodiments, the present invention relates to a method for treating central nervous system (CNS) related disorders, the method comprising administering to the patient a mesenchymal stem cell (MSC), an MSC-derived EV, or an MSC-derived bioxome, as described herein in detail, wherein said administration is intranasal administration to the patient.

In certain embodiments of the method for treating a CNS related disorder, the MSC, MSC-derived EV, or MSC-derived bioxome comprises an oncolytic virus or an oncolytic virus-based vector.

In certain embodiments of the method for treating a CNS related disorder, the MSC, MSC-derived EV, or MSC-derived bioxome comprises an oncolytic virus-based vector. In certain embodiments, the oncolytic virus-based vector is selected from the group consisting of an Adenovirus-based vector, an HSV-based vector, a VACV-based vector, a VSV-based vector, a Poliovirus-based vector, a Reovirus-based vector, a Senecavirus-based vector, an Echovirus-based vector, a SFV-based vector, a Maraba virus-based vector, and an Enterovirus-based vector.

In certain embodiments, the oncolytic virus-based vector is selected from the group consisting of ICOVIR-5 (Ad-DM-E2F-K-Δ24RGD), a Herpes simplex virus type 1 mutant 1716 (HSV-1716), Oncorine (H101), Onyx-15 (dl1520), ColoAd1, Talimogene laherparepvec (T-VEC), GL-ONC1, CV706, and GLV-1h68. In certain embodiments, the oncolytic virus-based vector is ICOVIR-5.

In certain embodiments of the method for treating a CNS related disorder, the MSC, MSC-derived EV, or MSC-derived bioxome comprises an oncolytic virus. In certain embodiments, the oncolytic virus is selected from the group consisting of a Herpes simplex virus (HSV), an Adenovirus, a Vaccinia virus (VACV), a Vesicular stomatitis virus (VSV), a Poliovirus, a Reovirus, a Senecavirus, an Echovirus, a Semliki Forest virus (SFV), a Maraba virus, and an Enterovirus.

In certain embodiments of the MSC, MSC-derived EV, or MSC-derived bioxome, the expression and activity of different genes and proteins is artificially inhibited in order to decrease the immunogenicity of the MSC. In certain embodiments of the MSC, MSC-derived EV, or MSC-derived bioxome, the MAVS gene or protein is inhibited.

A “CNS related disorder” or “CNS disease or disorder,” as used herein encompasses any condition that affects the brain and/or spinal cord and that leads to suboptimal function of an individual, including, for example, neurodegenerative disorders.

In certain embodiments the central nervous system (CNS) related disorders comprise Alzheimer's disease, Bell's palsy, epilepsy, cognitive aging, lewy body dementia, Parkinson's disease, cerebral palsy, motor neuron disease, multiple sclerosis (MS), neurofibromatosis, sciatica, shingles or any combination thereof.

In some embodiments, the CNS related disorder comprises Alzheimer's disease. In some embodiments, the CNS related disorder comprises Parkinson's disease. In some embodiments, the CNS related disorder comprises dementia. In some embodiments the CNS related disorder comprises vascular dementia. In some embodiments, the CNS related disorder comprises lewy body dementia. In some embodiments, the CNS related disorder comprises cognitive impairment. In some embodiments, the CNS related disorder comprises cognitive aging. In some embodiments, the CNS related disorder comprises Bell's palsy. In some embodiments, the CNS related disorder comprises cerebral palsy. In some embodiments, the CNS related disorder comprises epilepsy. In some embodiments, the CNS related disorder comprises motor neuron disease. In some embodiments, the CNS related disorder comprises multiple sclerosis (MS). In some embodiments, the CNS related disorder comprises neurofibromatosis. In some embodiments, the CNS related disorder comprises sciatica. In some embodiments, the CNS related disorder comprises shingles. In some embodiments, the CNS related disorder comprises a neurodegenerative disorder. In some embodiments, the CNS related disorder comprises a combination of neurodegenerative disorders.

In some embodiments, treating a CNS related disorder comprises ameliorating the symptoms of the CNS disorder. In some embodiments, treating a CNS related disorder comprises improving Alzheimer's Disease Assessment Scale (ADAS) score. In some embodiments, treating a CNS related disorder comprises improving delayed Rey Auditory Verbal Learning Test (dAVLT) score. In some embodiments, treating a CNS related disorder comprises improving delayed memory recall from the Wechsler Memory Scale scores. In some embodiments, treating a CNS related disorder comprises improving Digit Symbol Substitution score. In some embodiments, treating a CNS related disorder comprises improving the Trail Making Test part B score.

In some embodiments, treating a CNS related disorder comprises reducing the apolipoprotein E concentration in the brain. In some embodiments, treating a CNS related disorder comprises reducing the amyloid beta (AB) concentration in the brain. In some embodiments, treating a CNS related disorder comprises reducing t-tau concentration in the brain. In some embodiments, treating a CNS related disorder comprises reducing p-tau concentration in the brain.

The terminology used herein is for the purposes of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

Whenever the term “about,” is used, it is meant to refer to a measurable value such as an amount, a temporal duration, and the like, and is meant to encompass variations of ±25%, ±20%, ±10%, ±5%, ±1%, or ±0.1% from the specified value, as such variations are appropriate to perform the disclosed methods.

Throughout this application, various embodiments of this invention may be presented in a range format. It should be understood that, the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

### EXAMPLES

### Example 1. Intranasal Administration of MSCs Bearing an Oncolytic Virus or Oncolytic Virus-Based Vectors for Treating Cancer

Cancer patients are being treated by a first active agent, the first active agent being an oncolytic virus or an oncolytic virus-based vector. The first active agent is encapsulated and within a cell, for example a mesenchymal stem cell (MSC); an EV, for example an MSC-derived EV; or a bioxome, for example an MSC-derived bioxome.

Different oncolytic viruses or oncolytic virus-based vectors are known in the field, for example in Mol. Ther. 2007 September; 15 (9): 1607-15. doi: 10.1038/sj.mt.6300239. Epub 2007 Jun. 19; which is incorporated herein by reference in its entirety.

Cell-encapsulated oncolytic viruses or oncolytic virus-based vectors, and methods for producing them, are known in the field, for example in International PCT Application Publication WO 2019/043282, and Spanish Patent ES 2702618 B2, which are herein incorporated by reference in their entirety.

EV and bioxome-encapsulated oncolytic viruses or oncolytic virus-based vectors, and methods for producing them, are also known in the field, for example in International PCT Application Publication WO 2019/198068, which is herein incorporated by reference in its entirety.

Next, the encapsulated first active agent is administered intranasally to the patient. The cancer is brain cancer, for example glioblastoma, which may be a solid tumor, a primary tumor, or a metastatic tumor.

By being administered intranasally to the patient, the first active agent may reach the brain of the patient, for example applied to the olfactory nerve cells of the patient.

Several different methods and products are known for intranasal administration, for example devices such as instillation catheters, droppers, sprays, squeeze bottles, pump sprays, compressed air nebulizers, metered-dose inhalers, or insufflators. The intranasal administration device is optionally a vortical flow, controlled particle dispersion (CPD) device.

Methods and products are known for intranasal administration, for example in US Patents U.S. Pat. No. 7,231,919 B2 and U.S. Pat. No. 8,122,881, which are herein incorporated by reference in their entirety.

### Example 2. In Vitro Studies: Cytopathic Effect of MSCs and Conditioned Media on GBM Cell Line

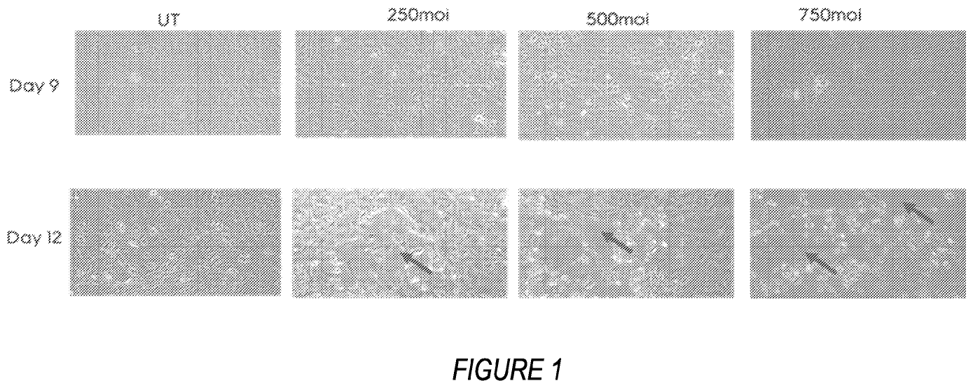

In vitro studies were performed to analyze the viral production and oncolytic effect of adenoviruses in the cells. The oncolytic virus infected mouse MSC WT cells inducing cell death and release of the virus into the culture media. While the cytopathic effect was detected only 12 days post viral infection (FIG. 1), the viral DNA could be detected as 5 days post infection (data not shown). The cytopathic effect and the viral DNA amount was correlated to the viral load.

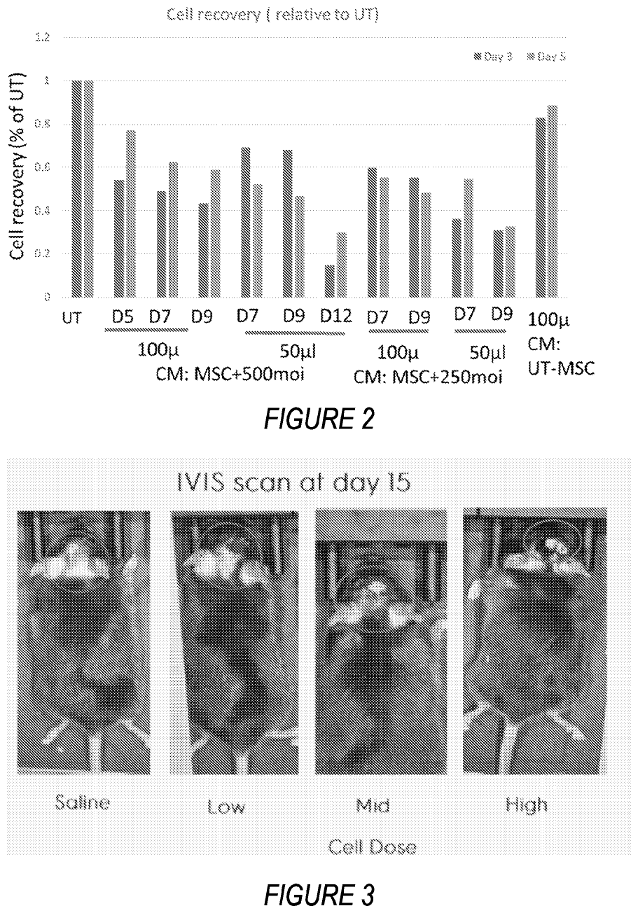

Additionally, the effect of the oncolytic virus bearing mouse mesenchymal stem cells was evaluated for the viability and survival of Glioblastoma cell line GL261 in vitro (FIG. 2).

### Example 3. Establishing GL261 Syngeneic Murine Glioblastoma Model

Murine glioblastoma model using GL261 cell line was successfully established. This mouse model is regarded as a gold standard and is widely used to investigate potential immunotherapies and other treatments. Mouse glioblastoma cell line GL261 with luciferase fluorescence was used. Three doses of cells were tested: Low dose: 0.1×10⁶ cells; Mid dose: 0.2×10⁶ cells; High dose: 0.5×10⁶ cells. The route of administration was Intracranially via stereotact (5 μl).

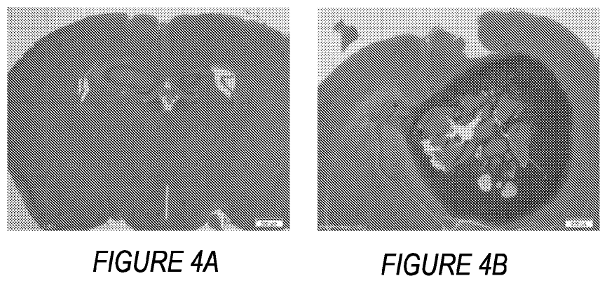

Results: Tumor cell expansion was detected via in-vivo imaging under a IVIS system (FIG. 3) and histology analysis (FIGS. 4A-4B). The majority of animals were positive for luciferase in the brain, three days after injection (detected by IVIS imaging).

The higher dose of 0.5×10⁶ cells yielded the most robust model and was used in subsequent studies.

### Example 4. Intranasal Administration of Murine MSCs

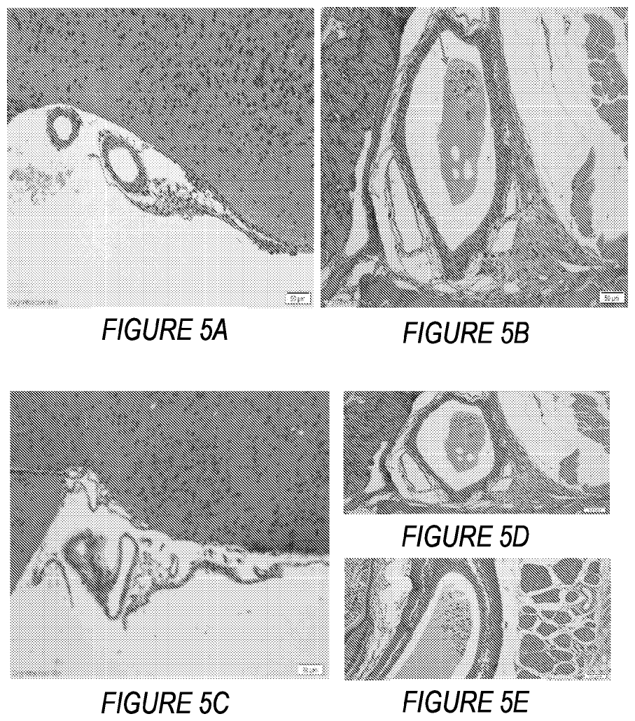

Single intranasal (IN) administration of wild-type murine MSCs was successful—MSCs were found in the maxillary sinus 24 h after administration as well as the ventral part of the mid brain, within the meninges along the blood vessels (FIGS. 5A-5E).

### Example 5. Pilot Study in Murine GBM Orthotopic Model (GL261)

A pilot study in murine GBM orthotopic model in C57bl/6 mice, using intranasal administration of MSCs via N2B device (experimental groups are shown in Table 1). Briefly, 48 hours after model establishment (glioblastoma GL261 luciferase labeled cells administered intracranially, as described in Example 3), mice were administered mouse MSCs bearing the oncolytic virus ICOVIR-5 (Celyvir) using the N2B device in 4 small 10 μl deliveries performed in succession, within one minute.

TABLE 1

| | | | | Dose | |
|---|---|---|---|---|---|
| | | GBM | | (cells/ | |
| Group | N | Model | Treatment | mouse) | Delivery |
| 1M | 3 | Mock | Vehicle (PBS) | — | IN |
| | | Intracranial | | | |
| | | (PBS - 5 μl) | | | |
| 2M | 8 | GL261-Luc | Vehicle (PBS) | — | IN (10 μl × 4) |
| 3M | 8 | 0.5 × 10⁶ | Control MSCs | 1 × 10⁶ | IN (10 μl × 4) |
| 4M | 8 | Intracranial | ICOVIR-5 | 1 × 10⁶ | IN (10 μl × 4) |
| | | (5 μl) | infected MSCs | | |
| 5M | 4 | | ICOVIR-5 | 1 × 10⁶ | IN (10 μl × 4) |
| | | | infected | | |
| | | | MAV−/− MSCs | | |

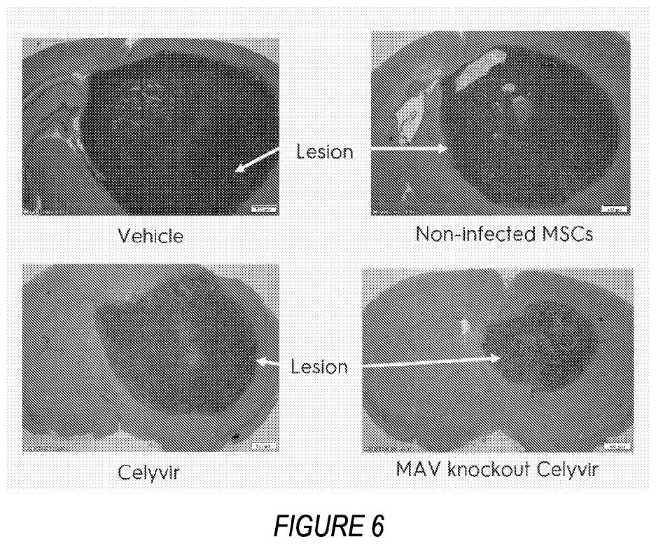

Results: 28 days post IN administration, a strong trend of tumor size reduction was demonstrated following a single administration of ICOVIR5-infected mMSCs, and a significant reduction of tumor size in the group treated with ICOVIR5-infected MAV knockout mMSCs (˜50% of reduction) in comparison to controls (FIG. 6).

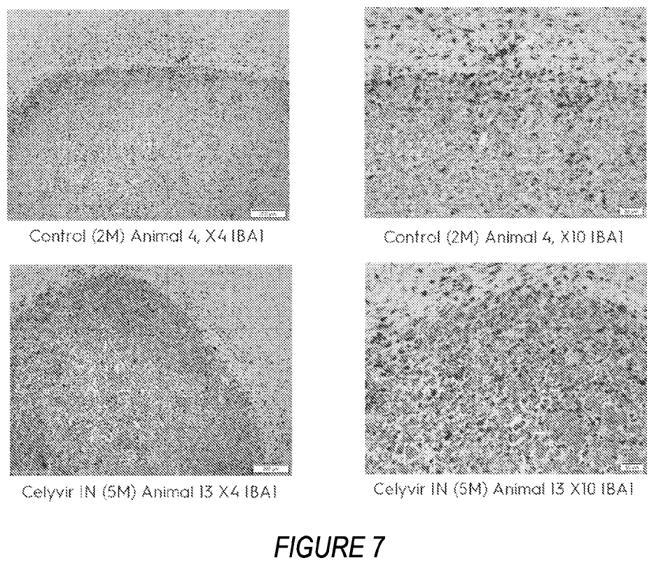

Macrophage infiltration was observed surrounding the tumor of animals treated with ICOVIR5-infected mMSCs (Celyvir) demonstrating active inflammation (anti-tumor activity). The CNS-associated immune stimulation was tested by Immunohistochemistry analysis of IBA-1, a calcium-binding protein expressed exclusively by Microglia in the CNS. A trend of Microglia infiltration into the GBM tumor lesion was demonstrated in ICOVIR5-infected mMSCs (Celyvir) IN treated groups in comparison to control groups (FIG. 7).

### Example 6. Biodistribution Pilot Study in Murine GBM Orthotopic Model (GL261)

In a pilot biodistribution study, mMSCs were pre-labeled with PKH26 fluorescent dye (one hour prior to intranasal administration) and mice were administered IN 72 h after GBM model establishment (glioblastoma GL261 luciferase labeled cells administered intracranially, as described in Example 3), (the experimental groups are shown in Table 2).

TABLE 2

| | | Dose | |
|---|---|---|---|
| Group | Treatment | (cells/mouse) | Delivery |
| 1M | Vehicle (PBS) (PKH26 label) | — | IN |
| 2M | MSCs (PKH26 label) | 1 × 10⁶ | IN |
| 3M | MAV−/− MSCs (PKH26 label) | 1 × 10⁶ | IN |

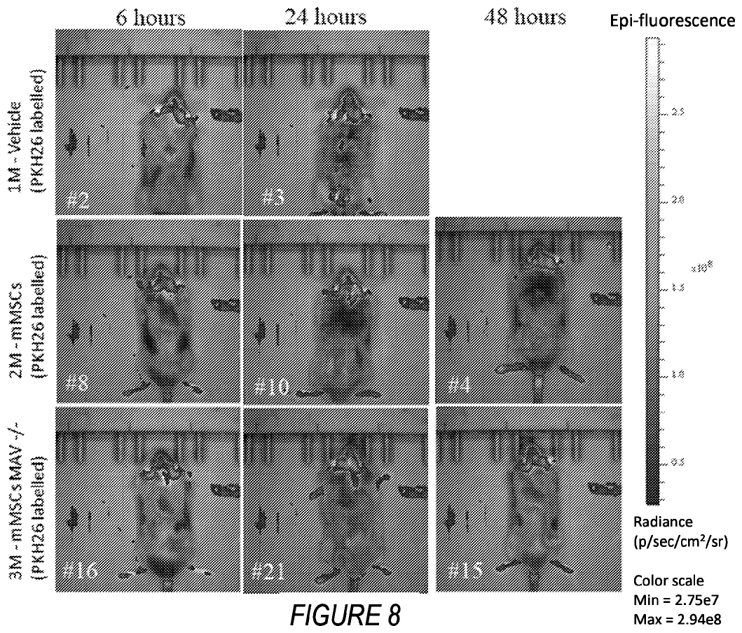

Animals were taken for analysis at 6, 24 and 48 hours post IN administration, 1 or 3 animals per time point. The fluorescent signal of PKH26 labelled MSCs was detectable by In Vivo Imaging System (IVIS) analysis from 6 up to 48 hours after IN administration (FIG. 8). Results demonstrated positive fluorescence signal of the cells in all tested groups with a considerable concentration in the tumor lesion area (where the GBM cells were injected).

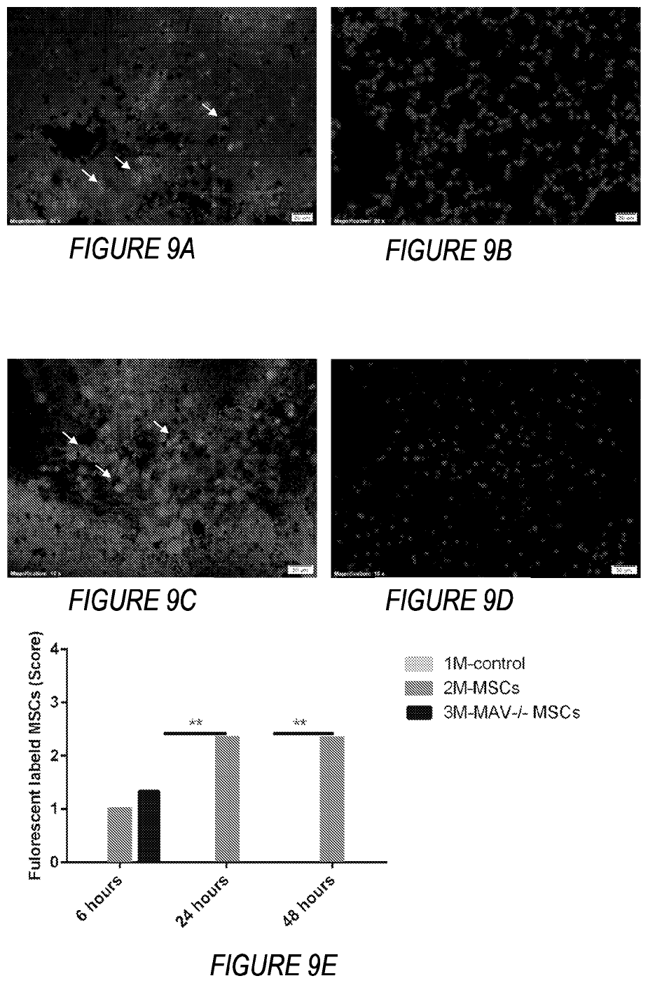

Brain sections and frontal sinuses were further analyzed to confirm localization of PKH26 labelled cells 6, 24 and 48 hours post IN administration. Histo-fluorescence analysis demonstrated PKH26 positive cells within the mucosal epithelium cells and within the brain (FIGS. 9A-9D) in both MSC treatment groups of mice IN treated with PKH26 labelled MSCs (Group 2M) and mice IN treated with PKH26 labelled MAV knockout MSCs (Group 3M). A further semi-quantitative analysis of these findings, using a scoring scale demonstrated the highest score (within the brain area) for PKH26 labeled MSCs (group 2M) versus control and MAV knockout MSCs (Group 3M) (FIG. 9E).

### Example 7. N2B Device Calibration and Optimization

MSCs were administered intranasally (IN) via N2B device. The first version of the device had a suction tube which delivered the test item into the nasal delivery aperture at a 90 degree angle. The second V2 device had a vacuum chamber connected to the delivery tube which directly delivered the test item to a narrow tube designed specifically for mice nose.

Using the first device iteration, no cells were detected in the nose or brain.

A new design was developed (V2 device), and cells could be detected in the nose and brain 24 h following IN administration.

No gross pathology nor histopathology detected.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

The invention claimed is:

1. A method for treating cancer in a patient, the method comprising the step of administering to the patient a first active agent, the first active agent selected from the group consisting of an oncolytic virus and an oncolytic virus-based vector, wherein the first active agent:
(i) is comprised in a mesenchymal stem cell (MSC), in an MSC-derived EV, or in an MSC-derived bioxome, and
(ii) is administered intranasally to the patient.

2. The method of claim 1, wherein the cancer is brain cancer.

3. The method of claim 2, wherein the brain cancer is glioblastoma.

4. The method of claim 1, wherein the cancer is a solid tumor, primary tumor, or metastatic tumor.

5. (canceled)

6. (canceled)

7. The method of claim 1, wherein the first active agent is an oncolytic virus-based vector or an oncolytic virus.

8. The method of claim 7, wherein the oncolytic virus-based vector is selected from the group consisting of an Adenovirus-based vector, an HSV-based vector, a VACV-based vector, a VSV-based vector, a Poliovirus-based vector, a Reovirus-based vector, a Senecavirus-based vector, an Echovirus-based vector, a SFV-based vector, a Maraba virus-based vector, and an Enterovirus-based vector.

9. The method of claim 7, wherein the oncolytic virus-based vector is selected from the group consisting of ICOVIR-5 (Ad-DM-E2F-K-Δ24RGD), a Herpes simplex virus type 1 mutant 1716 (HSV-1716), Oncorine (H101), Onyx-15 (dl1520), ColoAd1, Talimogene laherparepvec (T-VEC), GL-ONC1, CV706, and GLV-1h68.

10. The method of claim 9, wherein the oncolytic virus-based vector is ICOVIR-5.

11. (canceled)

12. The method of claim 7, wherein the oncolytic virus is selected from the group consisting of a Herpes simplex virus (HSV), an Adenovirus, a Vaccinia virus (VACV), a Vesicular stomatitis virus (VSV), a Poliovirus, a Reovirus, a Senecavirus, an Echovirus, a Semliki Forest virus (SFV), a Maraba virus, and an Enterovirus.

13. (canceled)

14. (canceled)

15. (canceled)

16. The method of claim 1, wherein in the MSC:
(i) a Toll-like receptor selected from the group consisting of TLR1, TLR2, TLR4, TLR5, TLR6, TLR7, TLR8, TLR9, and TLR10, gene or protein, is not inhibited,
(ii) the MyD88 gene or protein, is not inhibited, and/or
(iii) the MAVS gene or protein, is not inhibited.

17. The method of claim 1, wherein in the MSC:
(i) a Toll-like receptor selected from the group consisting of TLR1, TLR2, TLR4, TLR5, TLR6, TLR7, TLR8, TLR9, and TLR10, gene or protein, is inhibited,
(ii) the MyD88 gene or protein, is inhibited, and/or
(iii) the MAVS gene or protein, is inhibited.

18. The method of claim 17, wherein in the MSC, TLR4, TLR9, and/or the MyD88 gene or protein, is inhibited.

19. The method of claim 17, wherein in the MSC, the MAVS gene or protein, is inhibited

20. The method of claim 1, wherein the first active agent is administered intranasally to the brain of the patient.

21. The method of claim 20, wherein the first active agent is applied to the olfactory nerve cells of the patient.

22. (canceled)

23. The method of claim 1, wherein the first active agent is administered by a vortical flow, controlled particle dispersion (CPD) device.

24. A mesenchymal stem cell (MSC), wherein in the MSC the MAVS gene or protein or a gene or protein regulated by the MAVS gene or protein, is inhibited, an EV derived from the MSC, or a bioxome derived from the MSC.

25. (canceled)

26. A method for the production of a substantially pure population of mesenchymal stem cells according to claim 24, the method comprising the steps of:
(i) obtaining a sample of mesenchymal stem cells,
(ii) culturing the mesenchymal stem cells obtained in step (i), and
(iii) inhibiting the MAVS gene or protein, or of a gene or protein regulated by the MAVS gene or protein, in the cells cultured in step (ii).

27. A method for treating a central nervous system (CNS) related disorder, the method comprising administering to the patient a mesenchymal stem cell (MSC), an MSC-derived EV, or an MSC-derived bioxome, wherein said administration is intranasal administration to the patient.

28. The method of claim 27, wherein said MSC, MSC-derived EV, or MSC-derived bioxome comprises an oncolytic virus or an oncolytic virus-based vector.
